(19) United States
(12) Patent Application Publication
(10) Pub. No.: US 2025/0025594 A1
(43) Pub. Date: Jan. 23, 2025

(54) Frequency-Dependent Non-Thermal Plasma Inactivation Of Airborne Pathogens

(71) Applicant: The Regents of the University of Michigan, Ann Arbor, MI (US)

(72) Inventors: Herek L. CLACK, Ann Arbor, MI (US); Zhenyu MA, Ann Arbor, MI (US)

(21) Appl. No.: 18/770,859

(22) Filed: Jul. 12, 2024

Related U.S. Application Data
(60) Provisional application No. 63/527,428, filed on Jul. 18, 2023.

(51) Int. Cl. A61L 9/22

(52) U.S. Cl. CPC A61L 9/22; A61L 2209/14

(57) ABSTRACT

The degree of airborne virus inactivation, along with non-thermal plasma power, are dependent on the AC driving frequency used to energize the non-thermal plasma. As such, for AC-power non-thermal plasma air sterilization, there exists one or more frequencies at which dramatically enhanced pathogen inactivation can be achieved. The identification of the frequencies associated with peak pathogen inactivation, peak non-thermal plasma power (per volumetric flow rate), and peak ozone offers opportunities for optimization to achieve highest pathogen inactivation while consuming less power and producing less harmful ozone. Advanced non-thermal plasma-based air sterilization technologies could be based on systems designed with such optimization strategies.

### CROSS-REFERENCE TO RELATED APPLICATIONS

This application claims the benefit of U.S. Provisional Application No. 63/527,428, filed on Jul. 18, 2023. The entire disclosure of the above application is incorporated herein by reference.

### FIELD

The present disclosure relates to non-thermal plasma and, more particularly, relates to frequency-dependent non-thermal plasma inactivation of airborne pathogens.

### BACKGROUND AND SUMMARY

This section provides background information related to the present disclosure which is not necessarily prior art. This section provides a general summary of the disclosure, and is not a comprehensive disclosure of its full scope or all of its features.

Non-thermal plasmas (NTPs) used for sterilization and disinfection of flowing air streams offer a variety of advantages over other technologies for preventing the transmission of airborne infectious diseases through biological aerosols (bioaerosols) such as airborne viruses and bacteria.

Previous teachings typically presented the degree of airborne pathogen inactivation as a function of non-thermal plasma power (watts) delivered to the air flow, normalized by the air volumetric flow rate (e.g., liters per minute).

However, the present teachings disclose and demonstrate that the degree of airborne virus inactivation, along with non-thermal plasma power, are dependent on the AC driving frequency used to energize the non-thermal plasma. As such, for AC-power plasma air sterilization, there exists one or more frequencies at which dramatically enhanced pathogen inactivation can be achieved. The identification of the frequencies associated with peak pathogen inactivation, peak non-thermal plasma power (per volumetric flow rate), and peak ozone offers opportunities for optimization to achieve highest pathogen inactivation while consuming less power and producing less harmful ozone. Advanced non-thermal plasma-based air sterilization technologies could be based on systems designed with such optimization strategies.

According to an aspect of the present disclosure, a frequency-dependent non-thermal plasma system is provided for inactivation of airborne pathogens. The system includes a non-thermal plasma system configured to be driven by an alternating-current driving frequency. The alternating-current driving frequency energizing a non-thermal plasma of the non-thermal plasma system and being selected such that enhanced pathogen inactivation can be achieved.

According to a further aspect of the present disclosure the non-thermal plasma system is disposed within a tubular structure and further comprising a fan for directing air through the tubular structure.

According to a further aspect of the present disclosure the non-thermal plasma system includes a borosilicate bead packed bed surrounded by a high voltage electrode that is connected to a high voltage power supply.

According to a further aspect of the present disclosure, a pair of perforated brass plates are disposed on opposite sides of the borosilicate packed bed.

According to a further aspect of the present disclosure, each of the pair of perforated brass plates are electrically grounded.

According to a further aspect of the present disclosure, a pair of flow plugs are each disposed at a center of a respective one of the pair of perforated brass plates.

According to a further aspect of the present disclosure, an ozone filter is disposed in the tubular structure downstream from the borosilicate packed bed.

According to another aspect of the present disclosure, a frequency-dependent non-thermal plasma system for inactivation of airborne pathogens includes a tubular structure including a borosilicate bead packed bed surrounded by a high voltage electrode that is connected to a high voltage power supply configured to be driven by an alternating-current driving frequency. The alternating-current driving frequency energizing a non-thermal plasma within the borosilicate bead packed bed and being selected such that enhanced pathogen inactivation can be achieved. A fan draws air through the tubular structure.

According to a further aspect of the present disclosure, a pair of perforated brass plates are disposed on opposite sides of the borosilicate packed bed.

According to a further aspect of the present disclosure, each of the pair of perforated brass plates are electrically grounded.

According to a further aspect of the present disclosure, a pair of flow plugs are each disposed at a center of a respective one of the pair of perforated brass plates.

According to a further aspect of the present disclosure, an ozone filter is disposed in the tubular structure.

Further areas of applicability will become apparent from the description provided herein. The description and specific examples in this summary are intended for purposes of illustration only and are not intended to limit the scope of the present disclosure.

### BRIEF DESCRIPTION OF THE DRAWINGS

The drawings described herein are for illustrative purposes only of selected embodiments and not all possible implementations, and are not intended to limit the scope of the present disclosure.

FIG. 1 is a schematic illustration of the non-thermal plasma device according to the principles of the present teaching; and

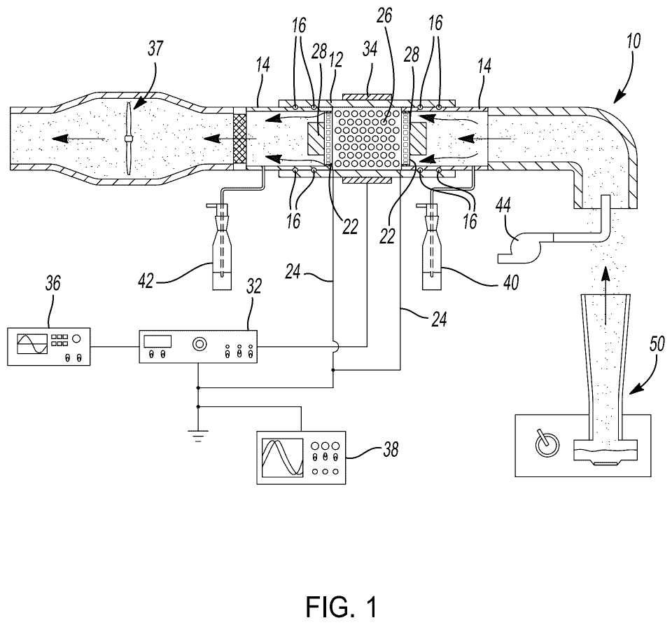

FIG. 2 is a flowchart for the operation of the non-thermal plasma device according to the principles of the present teaching.

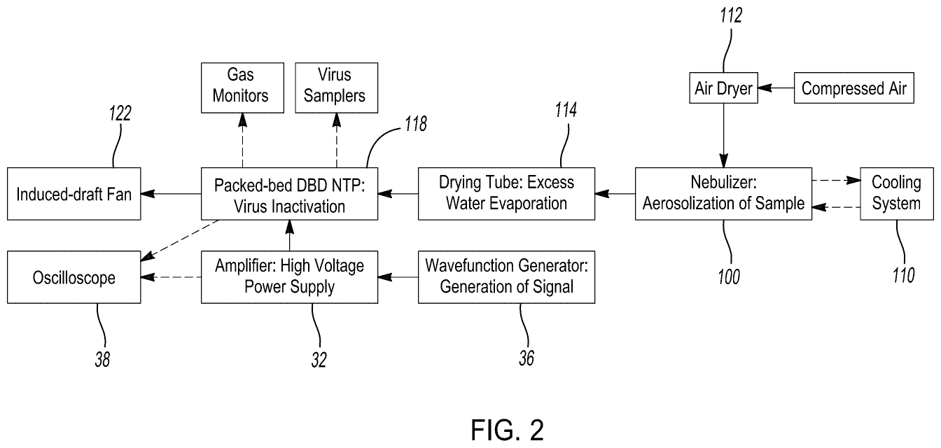

Corresponding reference numerals indicate corresponding parts throughout the several views of the drawings.

### DETAILED DESCRIPTION

Example embodiments will now be described more fully with reference to the accompanying drawings. Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The core setup of this dielectric barrier discharge (DBD) non-thermal plasma device 10 comprises a tubular structure including a system of cylindrical acrylic glass tubes 12, 14, including larger acrylic glass tube(s) 12 and two smaller acrylic glass tubes 14. The larger acrylic glass tube 12 can have an outer diameter of 4 inches and a length of 8 inches. The two smaller tubes 14 are able to slide into the larger one 12 and can each have an outer diameter of 3.5 inches and a length of 12 inches. Two pairs of rubber O-rings 16 sit around near the end 18 of the two smaller tubes 14 to ensure an air-tight sliding inside the larger tube 12. The two ends 20 of the smaller tubes 14 facing one another have perforated brass plates 22 affixed that serve as ground electrodes which are connected to ground wires 24. The perforated brass plates 22 hold in place a packed bed 26 of 500 borosilicate beads between them. Two cylindrical flow plugs 28 (diameter of 2.5 inches, height of 1 inch) made of acrylic glass are connected to the center of the two brass plates 22 inside the smaller tubes 14, to direct the airflow into an annular region 30 on the outer ring of the borosilicate beads packed bed 26. A brass ring 34 is positioned around the outer acrylic glass tube 12 at the packed-bed 26 region and is connected to an AC high-voltage power supply 32, to serve as the high-voltage electrode 34.

The tubes 12, 14 are made from a dielectric material to insulate the electrode 34 from the grounding plates 22. Although acrylic glass is disclosed as an example material for the tubes 12, 14, other dielectric materials can be used. In addition, although brass is disclosed as the material for the electrode ring 34 and the perforated grounding plates, other electrically conducting materials can be used. Further, although borosilicate beads have been used for the packed bed 26, other conductive and nonconductive materials and combinations of conductive and nonconductive materials can be used for the packed bed 26.

The high-voltage power supply 32 includes an amplifier 32 (Trek Model-610E) with an operating voltage ranging from 0 to 20 kV. The input to the amplifier 32 is generated from a function generator 36 (BK Precision Model-4052), which can output a sinusoidal waveform with a frequency ranging from 10 to 1000 Hz. An ozone filter 48 can be provided at a downstream location from the borosilicate beads packed bed 26 for filtering out ozone created by the non-thermal plasma.

For purposes of an experimental arrangement for determining the frequencies associated with peak pathogen inactivation, peak non-thermal plasma power (per volumetric flow rate), the signals of applied voltage and return current from the amplifier 34 can be monitored by an oscilloscope 38 (BK Precision Model-2190D), which is used to determine the non-thermal plasma status and its power consumption.

For the experimental arrangement, the inactivation of viral aerosols, MS2 aerosols are generated using an ultrasonic nebulizer 50 at step 100, which aerosolizes a bulk virus stock stored in the virus dilution buffer, at a rate of approximately 100 milliliters per hour. While aerosolizing, ice-cooled water is circulated in a metal tube in the nebulizer reservoir to keep its temperature from rising at step 110. The viral aerosols suspended in air are then mixed with compressed dry air from blower 44 at step 112, totaling an air flow rate of 200 lpm. The virus-laden air flow passes through a drying tube at step 114 to promote the evaporation of water from the aerosols. The drying tube has a length of 3 feet and a diameter of 3.5 inches, inside which a metal mesh separates the center airflow path with the surrounding annular region of desiccant material at step 116. After drying, the remaining viral aerosols are passed through the packed bed of borosilicate beads 26 at step 118 via one of the aforementioned smaller tubes 14 of the dielectric barrier discharge non-thermal plasma apparatus. The aerosols then make contact with the non-thermal plasma produced within the dielectric barrier discharge packed bed 26 in <0.5 s under typical conditions at step 120. An induced draft (ID) fan 37 is connected to the outlet end 38 of the packed bed 26 (the other smaller tube) at step 122, providing a flow of air through the entire system at the aforementioned rate of 200 lpm.

Virus samples are collected at both the inlet of the dielectric barrier discharge packed-bed 26 and the outlet using a pair of impingers 40, 42 (ACE Glass 7533-13) containing prepared virus dilution buffer. An electric vacuum pump (McMaster Carr model #4176K11) is turned on to draw samples from the airflow through the impingers 40, 42. Surviving viruses in the samples are then enumerated using the plaque-assay technique to back-calculate the number of plaque-forming units in the sample and thus to calculate that in the dielectric barrier discharge packed bed airflow. To rule out potential physical losses induced by the packed-bed non-thermal plasma design, the same experimental procedure is performed without power being supplied to the non-thermal plasma device 10 and the resulting virus samples are compared to the plasma inactivation results to confirm the effectiveness of the non-thermal plasma inactivation of airborne pathogens of the present disclosure.

The degree of airborne virus inactivation, along with non-thermal plasma power, are dependent on the AC driving frequency used to energize the non-thermal plasma. As such, for AC-power non-thermal plasma air sterilization, there exists one or more frequency ranges at which dramatically enhanced pathogen inactivation can be achieved. The identification of the frequencies associated with peak pathogen inactivation, peak non-thermal plasma power (per volumetric flow rate), and peak ozone level offers opportunities for optimization to achieve highest pathogen inactivation while consuming less power and producing less harmful ozone. Advanced non-thermal plasma-based air sterilization technologies could be based on systems designed with such optimization strategies.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A frequency-dependent non-thermal plasma system for inactivation of airborne pathogens, the system comprising:
a non-thermal plasma system configured to be driven by an alternating-current driving frequency, the alternating-current driving frequency energizing a non-thermal plasma of the non-thermal plasma system, the alternating-current driving frequency being selected such that enhanced pathogen inactivation can be achieved.

2. The frequency dependent non-thermal plasma system according to claim 1, wherein the non-thermal plasma system is disposed within a tubular structure and further comprising a fan for directing air through the tubular structure.

3. The frequency dependent non-thermal plasma system according to claim 2, wherein the non-thermal plasma system includes a borosilicate bead packed bed surrounded by a high voltage electrode that is connected to a high voltage power supply.

4. The frequency dependent non-thermal plasma system according to claim 3, further comprising a pair of perforated brass plates on opposite sides of the borosilicate packed bed.

5. The frequency dependent non-thermal plasma system according to claim 4, wherein each of the pair of perforated brass plates are electrically grounded.

6. The frequency dependent non-thermal plasma system according to claim 4, further comprising a pair of flow plugs each disposed at a center of a respective one of the pair of perforated brass plates.

7. The frequency dependent non-thermal plasma system according to claim 2, further comprising an ozone filter disposed in the tubular structure.

8. A frequency-dependent non-thermal plasma system for inactivation of airborne pathogens, the system comprising:
a tubular structure including a borosilicate bead packed bed surrounded by a high voltage electrode that is connected to a high voltage power supply configured to be driven by an alternating-current driving frequency, the alternating-current driving frequency energizing a non-thermal plasma within the borosilicate bead packed bed, the alternating-current driving frequency being selected such that enhanced pathogen inactivation can be achieved; and
a fan for drawing air through the tubular structure.

9. The frequency dependent non-thermal plasma system according to claim 8, further comprising a pair of perforated brass plates on opposite sides of the borosilicate packed bed.

10. The frequency dependent non-thermal plasma system according to claim 9, wherein each of the pair of perforated brass plates are electrically grounded.

11. The frequency dependent non-thermal plasma system according to claim 10, further comprising a pair of flow plugs each disposed at a center of a respective one of the pair of perforated brass plates.

12. The frequency dependent non-thermal plasma system according to claim 8, further comprising an ozone filter disposed in the tubular structure.
